(19) United States
(12) Patent Application Publication
(10) Pub. No.: US 2024/0325075 A1
(43) Pub. Date: Oct. 3, 2024

(54) ELECTROPORATION FOR SELECTIVE GI TRACT DEPTH ABLATION AND REGENERATION FOR GI DISEASES

(71) Applicant: Mayo Foundation for Medical Education and Research, Rochester, MN (US)

(72) Inventors: Barham K. ABU DAYYEH, Rochester, MN (US); Samuel J. ASIRVATHAM, Rochester, MN (US); William A. FAUBION, Rochester, MN (US); Jason A. TRI, Rochester, MN (US)

(21) Appl. No.: 18/660,908

(22) Filed: May 10, 2024

Related U.S. Application Data
(63) Continuation of application No. PCT/US2022/049653, filed on Nov. 11, 2022.
(60) Provisional application No. 63/278,707, filed on Nov. 12, 2021.

(51) Int. Cl. A61B 18/14; A61B 18/00

(52) U.S. Cl. CPC A61B 18/1492; A61B 2018/0022; A61B 2018/00482; A61B 2018/00577; A61B 2018/1435; A61B 2018/1467; A61B 2018/1475

(57) ABSTRACT

Endolumenal devices and methods can be used for the treatment of health conditions involving the different layers of the gastrointestinal tract. For example, this document describes devices and methods for treating diseases involving the gastrointestinal tract by endoscopically delivering pulsed electrical field or electroporation energy.

### BACKGROUND

### 1. Technical Field

This document relates to devices and methods for the treatment of health conditions involving the different layers of the gastrointestinal tract. For example, this document relates to devices and methods for treating diseases involving the gastrointestinal tract by delivering pulsed electrical field or electroporation endoscopically.

### 2. Background Information

The gastrointestinal tract comprises multiple layers including the mucosa, submucosal, muscularis propria, serosa or adventitia. It also includes deep or superficial nerves clusters referred to as the enteric nervous system and additional nerve ending surrounding the gastrointestinal tract. These different layers of the gastrointestinal tract and our surrounding structures are implicated and affected by different infectious, metabolic, inflammatory, or neoplastic diseases. Thus, gastrointestinal-based targeted therapies that can reach and treat these different layers selectively or comprehensively are desired but currently not achievable by thermal based modalities such as radiofrequency or microwave ablation.

Ablation therapy may be administered using probes inserted through the skin, through flexible tubes (catheters), inserted through a body conduit, or energy beams to reach the area being treated. Imaging techniques may be used to guide the ablation. The tissue is injured or destroyed with heat (e.g., radiofrequency ablation), extreme cold (cryoablation), lasers or a chemical.

### SUMMARY

This document describes devices and methods for the treatment of health conditions involving the different layers of the gastrointestinal tract. For example, this document describes devices and methods for treating diseases involving the gastrointestinal tract by delivering pulsed electrical field or electroporation endoscopically.

In one aspect, this disclosure is directed to an electroporation device that includes a shaft defining a lumen therethrough; an expandable balloon circumferentially attached about a distal portion of the shaft; and one or more electrodes that are selectively deployable to extend laterally from the balloon and that are completely retractable into the balloon.

In another aspect, this disclosure is directed an electroporation device that includes a shaft defining a lumen therethrough; an expandable frame circumferentially attached about a distal portion of the shaft; and one or more electrodes that are selectively deployable to extend laterally from the frame and that are completely retractable into the frame.

In another aspect, this disclosure is directed to an electroporation device that includes a cap configured to be releasably attached to a distal end portion of a shaft of an endoscopic device; and one or more electrodes that are selectively deployable to extend distally from the cap and that are completely retractable into the cap.

The electroporation devices described herein may optionally include one or more of the following optional features. The one or more electrodes may be curved electrodes. The one or more electrodes may be straight electrodes. The one or more electrodes may be helical electrodes. The electroporation devices may also include one or more flexible circuits attached to the balloon or the frame. The shaft may define a port that connects the lumen to areas external of the electroporation device. The balloon or the frame may define an opening configured for slidably receiving a guidewire.

In another aspect, this disclosure is directed to a method of administering pulsed electrical field electroporation energy to treat a disorder of a gastrointestinal tract of a patient. The method includes deploying any of the electroporation devices described herein to a target location within the gastrointestinal tract of the patient; deploying the one or more electrodes to extend laterally or distally and to penetrate into tissue at the target location; and energizing the one or more electrodes with pulsed electrical field electroporation energy to deliver the pulsed electrical field electroporation energy to the tissue at the target location.

Particular embodiments of the subject matter described in this document can be implemented to realize one or more of the following advantages. In some embodiments, methods and systems provided herein provide a minimally invasive therapy for various disorders of the gastrointestinal tract. For example, in some embodiments electroporation of the gastrointestinal mucosa is performed endoscopically. Such minimally invasive techniques can reduce recovery times, patient discomfort, and treatment costs. Additionally, the endoscopes, catheters, caps, and/or an over-tube as described herein can be used to ablate other portions of the gastrointestinal tract with both superficial and/or deep-pulsed electrical field for the treatment of various disorders of the gastrointestinal tract under endosonographic guidance.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

### DESCRIPTION OF THE DRAWINGS

FIG. 1 is a cross-sectional view of a portion of a human gastrointestinal tract.

FIG. 2 illustrates an example pulsed electrical field device for delivery of pulsed electrical field or electroporation endoscopically to the gastrointestinal tract.

FIG. 3 illustrates another example pulsed electrical field device for delivery of pulsed electrical field or electroporation endoscopically to the gastrointestinal tract.

FIG. 4 illustrates another example pulsed electrical field device for delivery of pulsed electrical field or electroporation endoscopically to the gastrointestinal tract.

FIG. 5 illustrates another example pulsed electrical field device for delivery of pulsed electrical field or electroporation endoscopically to the gastrointestinal tract.

Like reference numbers represent corresponding parts throughout.

### DETAILED DESCRIPTION

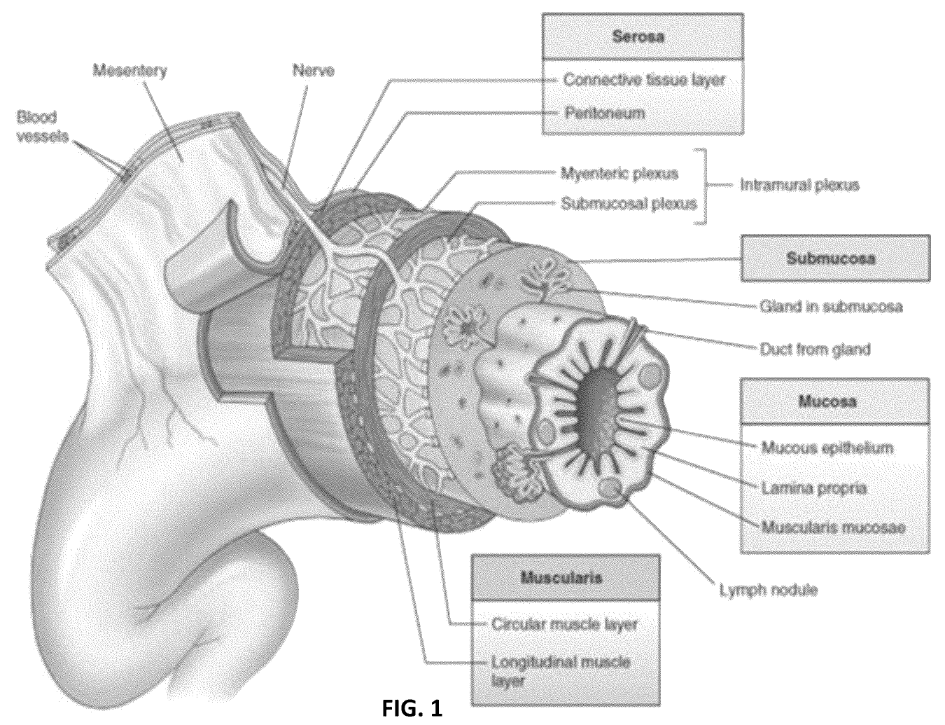

This document describes devices and methods for the treatment of health conditions involving the different layers of the gastrointestinal tract as shown FIG. 1, including the mucosa, submucosal, muscularis and the nerves within or surrounding the gastrointestinal tract in structures including the esophagus, stomach, small intestines, bile duct, pancreas duct, colon, rectum, and anal canal. For example, this document describes devices and methods for treating diseases involving the gastrointestinal tract by delivering pulsed electrical field or electroporation endoscopically.

Diseases treated by the devices and methods described herein can include, but are not limited to, flat or protruding polyps or masses of the gastrointestinal tract, Barrett's esophagus or esophageal cancer, metaplasia of the gastrointestinal tract, infiltrative disease of the gastrointestinal tract, lymphoma or other malignancies of the gastrointestinal tract, Helicobacter pylori infection, Clostridium difficile infection, or other infectious diseases of gastrointestinal tract, alteration of the gastrointestinal microbiome or leaky gut syndromes, irritable bowel disease, inflammatory bowel disease, eosinophilic GI diseases, celiac disease, hepatic encephalopathy, fatty liver disease, diabetes mellitus, achalasia, gastroparesis, Hirschsprung disease, deep GI malignancies invasion or invasion to neighboring lymph nodes, diseases requiring regeneration or ablation of the enteric nervous systems or surrounding nerve clusters to alter the gut-liver, gut-pancreas, gut-muscle, gut-adipose tissues, and or gut-central nervous system access. The devices and methods described herein allow partial or complete, selective or comprehensive ablation of the layers and or surrounding structures of the gastrointestinal tract.

Pulsed electrical field and/or electroporation therapy is a non-thermal energy delivery modality that can be used to ablate (e.g., to partially/temporarily affect cell walls or to completely terminate the living aspects of cells) and/or to regenerate living cells with minimal or no collateral thermal damage to other tissues. Currently, there are no endoscopic tools or accessories capable of delivering pulsed electrical field and/or electroporation therapy to the deeper wall structures of the gastrointestinal tract (e.g., such as submucosa, muscularis propria, nervous system, etc.) or surrounding structures.

The endoscopic tools and techniques described herein are capable of delivering both superficial and deep-pulsed electrical field directly to the gastrointestinal (“GI”) tract for the treatment of a variety of GI pathologies. In some embodiments, these tools can be guided endoscopically through the GI lumen through the working channel of an endoscope, and or over the endoscope as an overtube, and or adjacent to the endoscope. In addition, these tools can be guided to the appropriate depth of the GI or surrounding structures using optical white light only, filtered or processed light (narrow band imaging), and/or with endoscopic ultrasound guidance.

These tools (e.g., catheters, etc.), in conjunction with a generator, can emit and generate different pulse frequencies in the micro or nano range, and different voltage levels in the range of 100V-20 kV, for example. Different components of the same system (e.g., penetrating electrodes vs. superficial flexible electrodes) handle and generate/deliver (after connection to a generator) different voltages and pulse frequencies dependently or independently to achieve the desired treatment outcome.

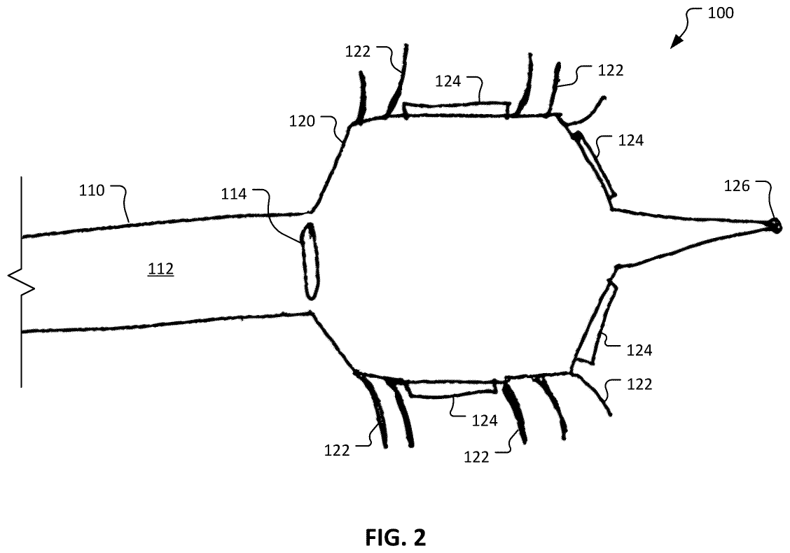

In one implementation as shown in FIG. 2, a pulsed electrical field device 100 includes a shaft 110 defining a first lumen 112 therethrough, and a balloon or expandable frame 120. The pulsed electrical field device 100 can be advanced to within the GI tract of a patient and used to deliver both superficial and deep-pulsed electrical field directly to the GI tract for the treatment of a variety of GI pathologies, as described herein.

In some embodiments, the balloon or expandable frame 120 can comprise a wire-form basket or framework. In some embodiments, the wire-form basket or framework is self-expanding (e.g., made of Nitinol or stainless steel). The balloon or expandable frame 120 is circumferentially attached about a distal portion of the shaft 110.

The shaft 110 has a hollow/open lumen 112 that can slidably accommodate various devices. For example, in some embodiments an endoscope, a suction device, and or an ultrasound probe for visualization, application of suction, and or application of additional energy or ablation modalities such as high frequency ultrasound can be advanced via the lumen 112 of the shaft 110. In some embodiments, a port 114 is defined by the shaft 110 or the balloon or expandable frame 120. The port 114 can be used to pass such devices from the lumen 112 to the exterior of the device 100 via the port 114. In some embodiments, more than one port 114 is included.

The balloon or expandable frame 120 includes multiple curved, straight, or helical penetrating electrodes 122 extending therefrom. The electrodes 122 are configured for penetrating the wall of the GI tract, for example. In some embodiments, all of the electrodes 122 are all of the same type (e.g., curved, straight, or helical). In some embodiments, two or more differing types of the electrodes 122 are included.

In some embodiments, some or all of the electrodes 122 have adjustable depths of penetration. That is, the electrodes 122 can be retracted into the balloon or expandable frame 120 and can be selectively deployed laterally from the balloon or expandable frame 120 (e.g., anywhere from zero to 3 cm, or more, in some examples) depending on the desired treatment location within the wall of the GI tract or surrounding structure.

In some embodiments, one or more of electrodes 122 can be needle electrodes that can be used to inject a substance (such as conductor, drug, target antibody, and/or immune modulator) into the desired target. In some embodiments, these needle electrodes 122 are contained within a sheath catheter that is flexible or steerable along the location of the balloon or expandable frame 120.

The adjustable penetration electrodes 122 can be deployed circumferentially to penetrate and treat the entire circumference of a portion of the GI tract, or can be condensed in one quadrant to treat part of the circumference of the portion of the GI tract. In some embodiments, one of more needle electrodes 122 can be assembled in tandem or parallel configuration along the length of the treatment area over the balloon or expandable shaft 120.

In some embodiments, the balloon or expandable frame 120 includes one or more flexible electronic circuits 124 mounted thereon. In some embodiments, the balloon or expandable frame 120 can have the one or more flexible circuits 124 to provide grounding or return electrodes, and/or to apply additional bipolar pulsed electric field as an additional complimentary or synergistic ablation or regeneration modality in addition to the penetrating electrodes 122. The flexible circuits 124 spacing, and designs can vary based on the target treatment area and desired depth of penetration within the GI tract or surrounding structures.

In some embodiments, the device 100 can be configured for advancement in the GI tract over a wire or a steerable visualization catheter using a distal opening 126.

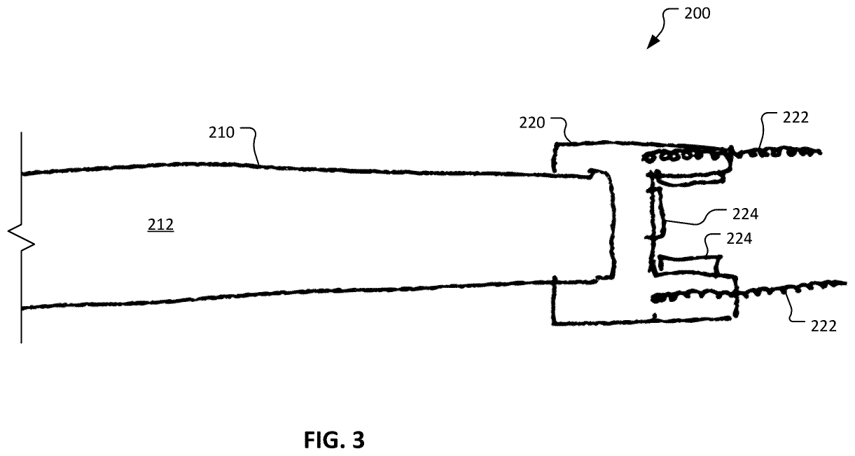

Referring to FIG. 3, another pulsed electrical field system 200 includes a shaft 210 and a cap 220 that is releasably coupled on a distal end portion of the shaft 210. The pulsed electrical field system 200 can be advanced to within the GI tract of a patient and used to deliver both superficial and deep-pulsed electrical field directly to the GI tract for the treatment of a variety of GI pathologies, as described herein.

In some embodiments, the shaft 210 is a forward-viewing or side-viewing instrument, such as endoscopic ultrasound device or other type of endoscopic instrument. The cap 220 is circumferentially coupled about a distal end portion of the shaft 210. The cap 220 can be releasably clamped or otherwise attached to the distal end portion of the shaft 210.

In some embodiments, the cap 220 includes one or more tissue penetrating straight, curved, or helical electrodes 222 that are selectively deployable from the cap 220. Mechanisms for deploying and adjusting the deployed length of the penetrating electrodes 222 can extend along the shaft 210 of the visualization instrument, and can be controlled by a clinician external of the patient.

The electrodes 222 can be deployed and/or controlled to point forward (distally) and/or sideways (radially or laterally) of the shaft 210 so as to apply the penetrating electrodes 222 in forward or side-ways configuration.

In some embodiments, one or more flexible circuits 224 are mounted on the luminal (non-penetrating) surface of the cap 220 to provide a return electrode configuration and or to apply bipolar-pulsed electrical field to the surface of the GI luminal structure to work in synergy with the penetrating electrodes 222. In a side-ways electrode deployment configuration, multiple penetrating electrodes 222 can be deployed and/or activated in sequence to treat a longer area of the GI tract. In some embodiments, the cap 220 contains one or more vacuum openings to allow apposition of target structure to the forward or side-cap. In some embodiments, the endoscope can provide visualization of the cap 220 and/or or suction to the cap 220.

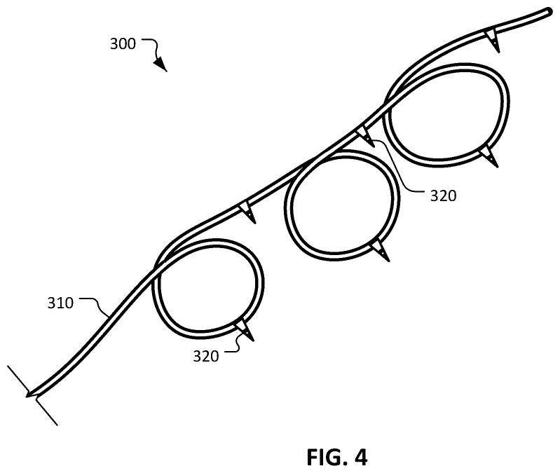

Referring to FIG. 4, another pulsed electrical field system 300 includes a catheter 310 and multiple weeping electrodes 320 attached to the catheter 310. The pulsed electrical field system 300 can be advanced to within the GI tract of a patient and used to deliver both superficial and deep-pulsed electrical field directly to the GI tract for the treatment of a variety of GI pathologies, as described herein.

The weeping electrodes 320 define one or more openings that are in fluid communication with a lumen of the catheter 310. Accordingly, a conductive fluid (or any type of fluid) supplied to the lumen of the catheter 310 can be exuded from the openings of the weeping electrodes 320. The weeping electrodes 320 have pointed tips that are configured to puncture into or indent tissue.

Although the pointed, indenting, and/or expandable roll-type electrodes 320 will significantly increase contact at varying depths from within the GI lumen into the tissue, this effect is significantly enhanced with irrigation at low flow from the points of maximum contact of a conducting solution such as normal saline, hypertonic saline, or similar solution. The electrical field size from the weeping electrodes 320 increases as a result of the virtual electrode phenomenon in which the conducting solution carries or conducts the electrical energy from the electrodes 320. Although there is a potential at a given electrical field strength for field density to decrease, importantly, because of the pressure of the pointed electrodes 320 (or reverse folded structure), there will tend to be permeation of the energy-conducting fluid into crypts, luminal vessels, etc., thus, counterbalancing the effect of increased overall field volume with focused concentration as a result of the convexity of the point and stasis of the virtual electrode within the tissue. The net result will be deeper lesions with relative superficial sparing.

A modification of this approach would be to use two different solutions for irrigation/weeping. The hypertonic saline or other conductive solution would be irrigated through the weeping electrodes 320 at the point(s) of maximal contact and indentation, etc., with hypotonic solution or a dielectric solution such as graphite, etc., or dextro solution with neither dielectric or conductive properties at sites on the electrodes and inter-electrode surfaces where there is minimal contact. The net result of this differential irrigation will be to focus electrical field density at deeper sites, minimizing superficial lesion creation.

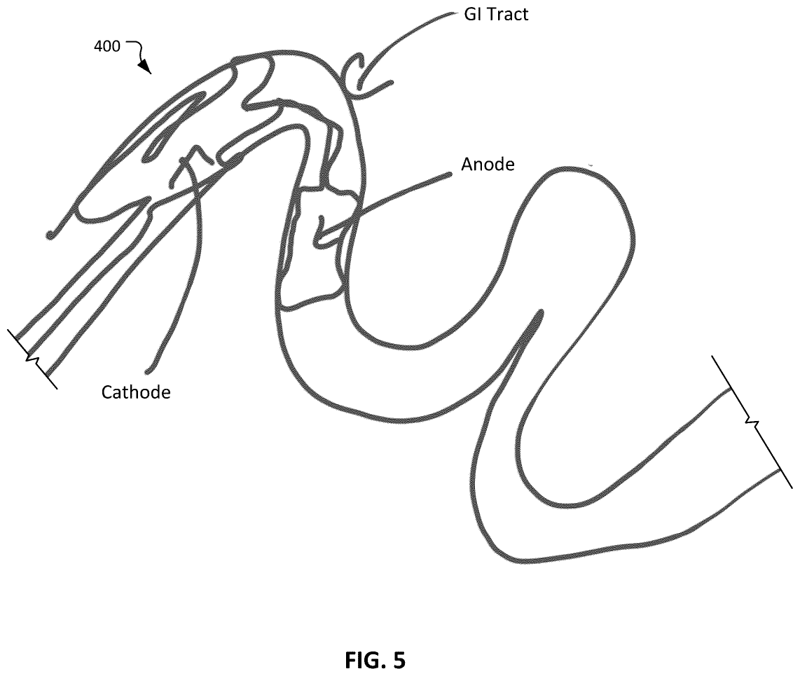

Referring to FIG. 5, in some embodiments the electrical field system 400 includes transmural bipolar electrodes. That is, the electrical field system 400 includes an electrode displaced endoluminally and another on the peritoneal surface. This, however, requires piercing the gut wall or entering the peritoneal space, both of which can be done and is indeed facilitated by these primary inventions. However, because of the potentially convolutional shaping and delectability of most parts of the GI tract, the electrical field system 400 involves placing two sets of electrodes-one at the target site serving as a sector or circumferential cathode and the second from the deflected site that opposes with the first in a u-shape type pattern serving as the anode. Specifically, the anodal layer will be of much larger surface area, minimizing effects at that site but in effect serving to drag the maximal electrical energy from the superficial lumen of the targeted site to the deeper layers.

### Additional Embodiments and/or Additional Features

In some embodiments, an adjunctive use of cryo-energy can be used. In such a case, the non-electroporation targeted sites can be brought into closer contact with the electrodes intended for delivering electroporation of pulsed DC field delivery, thus enhancing the effect of indentation. While this could be attempted with the use of vacuum or suction, the large surface area particularly of distended portions of the GI tract make this less attractive. Here, with or without suction, extendable-retractable electrodes are used for two purposes. At the target sites, the electrodes are extended and kept extended, indenting tissue and delivering pulsed direct current. In contradistinction, the electrodes at non-target sites are extended, cryo-energy delivered to cause tissue adherence, and the retracted so as to circumferentially or in a spiral manner, bring the tissue in closer contact to the primary ablating element and secondarily and specifically at sites of the protruding electroporation energy delivering electrodes.

In a variant of this iteration involving cryo-energy, two separate ablation delivering cylinders or scrolls are wrapped co-axially with the outer layer for cryo-energy that can be folded and deflected into the inner coil or inner spiral, maximizing contact, and then used for electroporation energy delivery. The cryo energy itself may have some additional electroporative benefit as a result of stretch, particularly when used with suction.

In some embodiments, neural and electromyographic recordings are used to serve as endpoints for energy delivery and automated titration. Although the electrodes are primarily intended for delivery of electrical energy, in this iteration of the invention, the electrodes either sequentially or simultaneously serve as the sensory limb of a potentially automated circuit. Signals including neural and those derived from smooth muscle action potentials have been shown to be recordable and with appropriate filtering including with the use of machine learning algorithms to iteratively filter contaminate cardiac signals and noise will recognize and register neural and myographic signals. With electroporation, there will be diminution of the signals as a result of ablated neural and smooth muscle tissue. However, the depth at which the lesion is being delivered can be deduced with predetermined algorithms by comparing electrograms at non-indenting electrode sites and indenting electrode sites. This equivalent of near and far-field bipolar electrograms can assess whether the lesion is superficial or at a targeted deeper plane. Because the electroporation signal can distort sensed electrograms with overwhelming noise and amplifier saturation, alternating electrodes, potentially in a phasic manner, can be used with separation of the circuit from the stimulatory channel to produce discernible electrograms both of human observation and automation.

In some embodiments, permittivity (e.g., the ability of a material to store electrical potential energy under the influence of an electric field measured by the ratio of the capacitance of a capacitor with the material as dielectric to its capacitance with vacuum as dielectric) is utilized. In some embodiments, the indenting electrodes are multilayered, with some exhibiting minimal indentation and others with deeper indentation. The intervening surface that slopes from one level of indentation to the other is also capable of delivering electrical pulses. Here, the more superficial and initial lesions are of low intensity so as to modify the impedance of the superficial layers. This creates a uniform but minimal field effect on the superficial layers so as to homogenize the impedance characteristics of this layer. This increases the permittivity of the electrical field, and the voltage gradient is shifted downstream to the edge of the homogenized impedance structures, i.e., the deeper layer. The deeper indenting electrodes now, both by virtue of their physician position and now with permittivity enhancement, more optimally delivers energy to the targeted deeper tissues.

In some embodiments, one or more deflectable telescoping electrodes can be included in the electrical field system. In this iteration, the electrodes are pre-mounted (as with a telescope) and then deployed where narrower portions are separated from broader, better-contacting portions. The entire telescoping network can be deflected with the aid of one or more pull wires so as to relatively appose the narrower and broader parts of the configuration. This configuration can promote facilitating deeper lesion formation and by varying the degree of apposition and the relative side of the anode and cathode.

In some embodiments, one or more helical electrodes can be included in the electrical field system. Although extendable retractable helices are used in stimulatory devices in a widespread manner, they are generally used to pierce the tissue and enter at deeper planes. While this could be done using any of the iterations invented here, damage to the mucosa, etc., would be undesirable. However, using a blunt, reverse tapering helix, no penetration will be made, but significant invagination and indentation into the area of interest, therefore increasing the surface area of contact and relative depth of the cathode would be facilitated.

In some embodiments, the electroporation devices and systems provided herein can include design features to prevent or inhibit undesired electro-stimulation of non-targeted bodily structures. For example, in some embodiments insulating elements can be included on or adjacent to one or more portions of the electroporation devices provided herein. Such insulating elements can block the emitted energy from following particular paths so as to protect non-targeted bodily structures. In some embodiments, insulated bipolar electroporation is incorporated (e.g., where the electrodes are mounted within or on a balloon, and/or separate electrodes are placed in the GI tract). Such electrodes can be used as the anode or cathode when the complimentary cathode or anode are located within, on, or as a separate electrode to a balloon placed in the GI tract. For example, the insulation can be an insulating coating on a particular side of a balloon, a second balloon which is insulated, or an air sac acting as insulation element to cover one side of the external surface of a balloon. In some embodiments, such insulating techniques can be used to cover one side of the external surface of a balloon. In some embodiments, bipolar electrodes are included (e.g., a distal electrode and a proximal electrode on an electroporation device).

In some cases when the aforementioned insulation is included, because of the insulation on the

Additionally, the devices and techniques described herein can be applied in contexts such as, but not limited to, the duodenum, the mucosa of the distal small and large intestines, and other endoluminal organs such as the gallbladder, pancreas, and in the arteriovenous system.

Additionally, the devices and techniques describes herein can be applied to pherese drugs to cells within the mucosa of the GI tract to alter their function. For example drugs such as rapamycin know to modulate the effects of paneth and stem cells within the crypts of the small intestines can be ionized and pheresed into these cell using electroporation. Furthermore, sweet substances known to stimulate the enteroendocrine cells within the villi of the duodenum can be applied. Similarly, tacrolimus can be used to stimulate stem cells in some cases. As such, these devices and techniques may cycle energy alone, drug or substance alone, or in combination to treat various conditions as described herein.

Some of the devices and methods provided herein can also incorporate stimulatory electrodes or other devices that can be used to ascertain cell death or activity, or to measure the temperature, electrical field strength, and/or charge density of the delivered electroporative therapy.

Some of the devices provided herein which incorporate a balloon or balloon-like elements may be used to achieve stretch of the intestine, not only to increase the surface area of contact to the crypt cells, but by virtue of the stretch itself produce membrane poration and induced apoptosis.

Some embodiments of the balloon or mesh incorporated devices are designed to increase the charge density of delivery through injection-like ports that may be achieved by a serrated surface or actual expandable, low surface area, pointed elements. These may serve as actual injection ports for charge or an electrolyte-rich solution to transfer the electroporation rendering energy or serve as regions of high electron or other electrical force density by virtue of their shape, which would match the required area where the increased density of charge is required and thus minimizing risks of electrical or thermal injury to the non-targeted sites.

It should be understood that one or more of the features described anywhere herein may be combined with one or more other features described anywhere herein to create hybrid devices and/or methods, without departing from the scope of this disclosure.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described program components and systems can generally be integrated together in a single product or packaged into multiple products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In certain implementations, multitasking and parallel processing may be advantageous.

What is claimed is:

1. An electroporation device comprising:
a shaft defining a lumen therethrough;
an expandable balloon circumferentially attached about a distal portion of the shaft; and
one or more electrodes that are selectively deployable to extend laterally from the balloon and that are completely retractable into the balloon.

2. An electroporation device comprising:
a shaft defining a lumen therethrough;
an expandable frame circumferentially attached about a distal portion of the shaft; and
one or more electrodes that are selectively deployable to extend laterally from the frame and that are completely retractable into the frame.

3. The electroporation device of claim 1 or claim 2, wherein the one or more electrodes are curved electrodes.

4. The electroporation device of claim 1 or claim 2, wherein the one or more electrodes are straight electrodes.

5. The electroporation device of claim 1 or claim 2, wherein the one or more electrodes are helical electrodes.

6. The electroporation device of claim 1 or claim 2, further comprising one or more flexible circuits attached to the balloon or the frame.

7. The electroporation device of claim 1 or claim 2, wherein the shaft defines a port that connects the lumen to areas external of the electroporation device.

8. The electroporation device of claim 1 or claim 2, wherein the balloon or the frame defines an opening configured for slidably receiving a guidewire.

9. An electroporation device comprising:
a cap configured to be releasably attached to a distal end portion of a shaft of an endoscopic device; and
one or more electrodes that are selectively deployable to extend distally from the cap and that are completely retractable into the cap.

10. The electroporation device of claim 9, further comprising one or more flexible circuits attached to the cap.

11. The electroporation device of claim 9, wherein the one or more electrodes are curved electrodes.

12. The electroporation device of claim 9, wherein the one or more electrodes are straight electrodes.

13. The electroporation device of claim 9, wherein the one or more electrodes are helical electrodes.

14. A method of administering pulsed electrical field electroporation energy to treat a disorder of a gastrointestinal tract of a patient, the method comprising:
deploying the electroporation device of any one of claims 1 through 13 to a target location within the gastrointestinal tract of the patient;
deploying the one or more electrodes to extend laterally or distally and to penetrate into tissue at the target location; and
energizing the one or more electrodes with pulsed electrical field electroporation energy to deliver the pulsed electrical field electroporation energy to the tissue at the target location.
